(19) United States
(12) Patent Application Publication
(10) Pub. No.: US 2026/0233264 A1
(43) Pub. Date: Aug. 13, 2026

(54) RETRACTABLE ALCOHOL TESTER WITH INTELLIGENT RECOGNITION

(71) Applicant: Shenzhen Dachenwei Technology Group Co., Ltd, Shenzhen (CN)

(72) Inventors: Changxing Li, Shenzhen (CN); Shenghui Hu, Shenzhen (CN); Rong Pan, Shenzhen (CN)

(21) Appl. No.: 19/263,679

(22) Filed: Jul. 9, 2025

(30) Foreign Application Priority Data
Feb. 13, 2025 (CN) 202510158174.9

(51) Int. Cl. B08B 9/032; G01N 33/497

(52) U.S. Cl. CPC B08B 9/0323; G01N 33/4972; B08B 2209/032; B08B 2230/01

(57) ABSTRACT

An alcohol tester includes a machine body assembly provided with a detection assembly for an alcohol detection, a cleaning assembly including an air heating component arranged in the detection assembly, a steaming assembly arranged above the air heating component and a triggering assembly arranged in the detection assembly, and a re-cleaning assembly including a shifting component arranged at the bottom of the detection assembly and a dividing assembly arranged below the shifting component. Before an alcohol detection, the machine body assembly recognizes a body size of a tested individual and the detection assembly moves approach the tested individual. After the alcohol detection, the air heating component blows hot air into the detection assembly, and the steaming assembly injects water into the air heating component to form steam, the shifting component intercepts pipelines of the alcohol detection, and the dividing assembly clean the pipelines of the alcohol detection.

### CROSS-REFERENCE TO RELATED APPLICATIONS

This application claims priority to Chinese patent application No. 202510158174.9 filed Feb. 13, 2025 which is incorporated by reference herein in its entirety for all purposes.

### TECHNICAL FIELD

The present disclosure relates to the technical field of alcohol testers, specifically relates to a retractable alcohol tester with intelligent recognition.

### BACKGROUND

With the improvement of people's traffic safety awareness, alcohol testers are widely used in various driving inspections, public safety and personal health monitoring. Traditional alcohol testers meet the basic requirements of breath alcohol concentration detection to a certain extent, but their design limitations have gradually become apparent, especially in terms of hygiene, convenience, and accuracy.

Firstly, considering the different exhalation habits and physical characteristics of different individuals, the blowing nozzle arranged in a fixed position is difficult to meet the requirements of all tested individuals. In particular, for the elderly or people with special physical conditions, a device that can intelligently recognize and automatically adjust the position and size of the blowing nozzle according to the individual's situation will greatly improve comfort and convenience.

Secondly, the design of blowing nozzle of the traditional alcohol testers is generally fixed and not allowed to be replaced or disassembled, which not only increases the risk of cross-infection among the tested individuals but also makes it difficult to thoroughly clean and disinfect the inside of the blowing nozzle after long-term use, resulting in the accumulation of dirt and bacteria.

Finally, the pipeline of the traditional alcohol tester generally retains the gas left during the previous testing. The residual gas may not only contain the exhalation components of the previous individual but also undergo physical or chemical changes due to the environmental humidity and temperature. Therefore, the residual gas adversely affects the data of the next testing and reduces the accuracy and reliability of the test result.

### SUMMARY

According to the disclosure, an alcohol tester is provided to solve at least one technical problem in the prior art. In addition, the alcohol tester with intelligent recognition performs high-temperature and steam disinfection on the blowing part after alcohol detection and cleans the gas delivery pipeline, effectively volatilizing residual alcohol and the bacteria left during blowing, thereby avoiding the impact of residual alcohol gas on the accuracy of subsequent tests and avoiding the cross-infection.

According to a first aspect of the present disclosure, an alcohol tester includes: a main body having a machine body assembly provided with a detection assembly for an alcohol detection at the bottom; a cleaning assembly including an air heating component arranged in the detection assembly, a steaming assembly arranged above the air heating component, and a triggering assembly arranged at the top of an inner cavity of the detection assembly; and a re-cleaning assembly including a shifting component arranged at the bottom of the detection assembly, and a dividing assembly arranged below the shifting component. Before an alcohol detection, the machine body assembly recognizes a body size of a tested individual and the detection assembly moves approach the tested individual. After the alcohol detection, the air heating component blows hot air into the detection assembly, and the steaming assembly injects water into the air heating component to form steam, the shifting component intercepts pipelines of the alcohol detection, and the dividing assembly clean the pipelines of the alcohol detection.

With reference to the first aspect, in an embodiment, the machine body assembly includes a main frame, a first camera fixed to the front top of the main frame, and an LED panel fixed to the front top of the main frame.

With reference to the first aspect, in an embodiment, the detection assembly includes: an electric drive component hinged at the bottom of front side of the main frame; an electric push rod mounted in the electric drive component; and a detection component connected to the top end of the electric push rod; wherein, the front side of the detection component is hinged with a sealing cover and a blowing nozzle is detachably connected to the front side of the detection component, and wherein, the detection component is provided with a locking component arranged above the blowing nozzle and a second camera is mounted at the top of the front side of the detection component.

With reference to the first aspect, in an embodiment, the air heating component includes: a thermal insulation gas buffer fixed to the top of an inner cavity of the detection component; at least one electric heating element mounted in the thermal insulation gas buffer; a first fan having an inlet and an outlet connected to the rear side of the thermal insulation gas buffer; an air inlet hood connected to the inlet of the first fan; and a blowing hood arranged above the blowing nozzle; wherein, the air inlet hood is fixed on the top surface of the detection component, the front side of the thermal insulation gas buffer is connected to one end of an air pipe, and the blowing hood is connected to the other end of the air pipe.

With reference to the first aspect, in an embodiment, the steaming assembly includes a water tank fixed to the top of the inner cavity of the detection component, a water pump connected to an outlet of the water tank, a water pipe connecting the water pump with the thermal insulation gas buffer, and a check valve mounted at a position where the thermal insulation gas buffer is connected to the water pipe.

With reference to the first aspect, in an embodiment, the triggering assembly includes: a sliding plate having an outward surface facing an outer side of the detection component and an inward surface facing an inner side of the detection component; a pressing rod connected to the outward surface of the sliding plate; a spring having one end connected to the inward surface of the sliding plate; and a first button mounted at the top of the inner cavity of the detection component; wherein, the sliding plate is slidably arranged at the top of the inner cavity of the detection component, and an outer end of the pressing rod extends out of the detection component in contact with the bottom of the sealing cover, the other end of the spring is fixed to the inner cavity of the detection component, and the first button is accessible to the outward surface of the sliding plate.

With reference to the first aspect, in an embodiment, the shifting component includes: a rotating frame including a base portion, a first arm, and a second arm having an sleeve portion for connecting pipelines for alcohol detection; a motor having an output shaft connected to the base portion of the rotating frame; an L-shaped pipe channel connecting the outer of the first arm to the base portion; and a second button mounted at the bottom of the inner cavity of a detection component in the detection assembly, wherein the second button is triggered by the second arm to clean the sleeve portion, and wherein the motor is mounted at the bottom of the inner cavity of the detection component.

With reference to the first aspect, in an embodiment, the dividing assembly includes: a three-way component having an inlet port, a first outlet port and a second outlet port; a second fan connected to the inlet port of the three-way component; a soft pipe having one end connected to the first outlet port of the three-way component, and the other end connected to the L-shaped pipe channel of the rotating frame; a blowing pipe having an inlet end connected to the second outlet port of the three-way component, and an outlet end for accessing to one side of the sleeve portion of the rotating frame; and an exhaust pipe having an inlet end for accessing to the other side of the sleeve portion of the rotating frame, and an outlet extending out of the detection component from the bottom; wherein the second fan is mounted at the bottom of the inner cavity of the detection component.

With reference to the first aspect, in an embodiment, the blowing pipe and the exhaust pipe are hard pipes made of brass.

With reference to the first aspect, in an embodiment, the second button is a switch for controlling an operation of the second fan.

With reference to the first aspect, in an embodiment, the rotating frame is provided with a hollow pipe in the sleeve portion, and the inner diameter of the hollow pipe matches with inner diameters of pipelines in the detection component.

According to a second aspect of the present disclosure, an alcohol tester includes: a main body having a machine body assembly provided with a detection assembly for an alcohol detection at the bottom; and a re-cleaning assembly including a shifting component arranged at the bottom of the detection assembly, and a dividing assembly arranged below the shifting component; wherein, after the alcohol detection, the shifting component intercepts pipelines of the alcohol detection, and the dividing assembly clean the pipelines of the alcohol detection. The shifting component includes: a rotating frame including a base portion, a first arm, and a second arm having an sleeve portion for connecting pipelines of the alcohol detection; a motor having an output shaft connected to the base portion of the rotating frame; an L-shaped pipe channel connecting the outer of the first arm to the base portion; and a button mounted at the bottom of the inner cavity of a detection component in the detection assembly, wherein the button is triggered by the second arm of the rotating frame rotated by the motor to clean the sleeve portion.

With reference to the second aspect, in an embodiment, the rotating frame is provided with a hollow pipe in the sleeve portion, and the inner diameter of the hollow pipe matches with inner diameters of pipelines in the detection component, and wherein, the hollow pipe connects the pipelines when the rotating frame is driven by the motor to rotate to a position for alcohol detection.

With reference to the second aspect, in an embodiment, the dividing assembly includes: a three-way component having an inlet port, a first outlet port and a second outlet port; a fan connected to the inlet port of the three-way component; a soft pipe having one end connected to the first outlet port of the three-way component, and the other end connected to the L-shaped pipe channel of the rotating frame; a blowing pipe having an inlet end connected to the second outlet port of the three-way component, and an outlet end for accessing to one side of the sleeve portion of the rotating frame; and an exhaust pipe having an inlet end for accessing to the other side of the sleeve portion of the rotating frame, and an outlet extending out of the detection component from the bottom; wherein the fan is mounted at the bottom of the inner cavity of the detection component.

The technical effects achieved by the present disclosure are as follows.

The machine body assembly and detection assembly in the present disclosure can record the facial features of the tested individual and adjust the position and height of the detection assembly to accommodate people of different body sizes. Before the detection, the camera of the machine body assembly records the facial features of the tested individual and detects the body size. Subsequently, the retractable part of the detection assembly rotates outward and extends to allow the detection part of the detection assembly to adapt to the body size of the tested individual. Afterward, the detection assembly is turned on for the detection. If the blowing nozzle needs to be replaced, it can be detached by lifting and pulling outward, thereby facilitating replacement after long-term use.

The air heating component, steaming assembly, and triggering assembly in the present disclosure can perform high-temperature disinfection and steam disinfection on the blowing part after the alcohol detection, which can effectively volatilize the residual alcohol and the bacteria left during blowing. Before the alcohol detection, when the detection assembly is turned on, the rebound part of the triggering assembly automatically unlocks and triggers the switch, causing the heating part of the air heating component to start preheating. After finishing the detection, when the camera of the detection assembly detects that the tested individual has left, the blowing part of the air heating component operates to blow air which is heated by the heating part to perform high-temperature sterilization on the blowing part of the detection assembly. At the same time, the delivery part of the steaming assembly injects purified water into the air heating component to form steam, such that the steam can be also disinfected during the high-temperature disinfection, thereby preventing the spread of bacteria and avoiding residual alcohol gas affecting subsequent detections.

The shifting component and dividing assembly in the present disclosure can prompt to clean the gas delivery pipeline after achieving the detection, thereby avoiding the impact of residual alcohol gas on the accuracy of subsequent detections. During the detection, the connection part of the shifting component guides the gas into the interior of the detection assembly for the detection. After achieving the detection, the shifting component is driven to rotate by a driving part thereof, causing the connection part to rotate downward and trigger the switch of the dividing assembly. At the same time, the blowing part of the shifting component is aligned with the blowing part of the detection assembly, and the dividing assembly operates to blow air, and a diversion part of the dividing assembly divides the blowing air into two streams. One stream of blowing air is blown into the blowing part of the detection assembly so as to flush the pipeline, while the other stream of blowing air enters the connection part of the shifting component to ensure the cleanliness of the internal air, thereby preventing the residual alcohol and the accumulation of bacteria that can affect subsequent detections and the safety of the device.

### DESCRIPTION OF DRAWINGS

FIG. 1 is a schematic diagram of an alcohol tester according to an embodiment of the present disclosure;

FIG. 2 is a schematic diagram of a machine body assembly of the tester according to the embodiment of the present disclosure;

FIG. 3 is a schematic diagram of the detection assembly according to an embodiment of the present disclosure;

FIG. 4 is a schematic diagram of the blowing nozzle according to an embodiment of the present disclosure;

FIG. 5 shows a sectional view of the detection component according to an embodiment of the present disclosure;

FIG. 6 is a schematic diagram showing the position of the locking component according to an embodiment of the present disclosure;

FIG. 6a shows an enlarged view of the structure between the blowing nozzle and the locking component;

FIG. 7 is a schematic diagram of the air heating component according to an embodiment of the present disclosure;

FIG. 8 is a schematic diagram of the steaming assembly according to an embodiment of the present disclosure;

FIG. 9 shows an exploded view of the triggering assembly according to an embodiment of the present disclosure;

FIG. 10 is a schematic diagram of the position of the shifting component according to an embodiment of the present disclosure;

FIG. 11 is a schematic diagram of the shifting component according to an embodiment of the present disclosure;

FIG. 12 shows a sectional view of the L-shaped pipe channel according to an embodiment of the present disclosure;

FIG. 13 is an exploded view of the dividing assembly according to an embodiment of the present disclosure, wherein the rotating frame is shown by sectioning;

FIG. 14 shows a sectional view of the rotating frame and L-shaped pipe channel according to an embodiment of the present disclosure;

FIG. 15 shows an enlarged view of the shifting component according to an embodiment of the present disclosure, wherein the rotating frame is rotated to a first position for gas detection; and

FIG. 16 shows an enlarged view of the shifting component according to an embodiment of the present disclosure, wherein the rotating frame is rotated to a second position for gas cleaning.

The disclosure is described in detail with reference to the drawings. In order to describe clearly the embodiments of the present disclosure, the cross-sectional views of the device structure may be partially enlarged without following general proportions. The schematic diagrams are only illustrative and should not limit the scope of protection of the present disclosure. In addition, the actual production should include three-dimensional spatial dimensions of length, width, and depth.

### DETAILED DESCRIPTION

The following will provide a clear and complete description of the concept, specific structure, and technical effects of the disclosure, with the help of embodiments and accompanying drawings, in order to fully understand the purpose, solution, and effects of the disclosure. It should be noted that, unless conflicting, the features in the embodiments of this application can be combined with each other.

Furthermore, unless otherwise specified, when a feature is referred to as “fixed” or “connected” to another feature, it can be directly or indirectly fixed or connected to the other feature. Additionally, the terms “up,” “down,” “left,” “right,” “top,” “bottom,” and similar descriptions used in this disclosure are relative to the positional relationship between the components of the disclosure as depicted in the drawings. The terms “an embodiment” or “embodiments” refer to specific features, structures, or characteristics included in at least one implementation of the disclosure. Moreover, unless otherwise defined, all technical and scientific terms used in this document have the same meaning as commonly understood by those skilled in the art.

It should be understood that, although the terms “first,” “second,” “third,” etc., may be used to describe various components in this disclosure, these components should not be limited to these terms. These terms are used to distinguish components of the same type from each other. For example, without departing from the scope of this disclosure, the first component may also be referred to as the second component, and similarly, the second component may also be referred to as the first component.

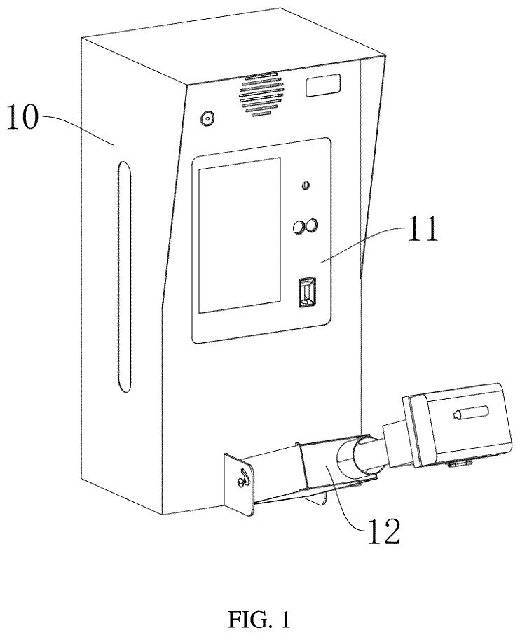

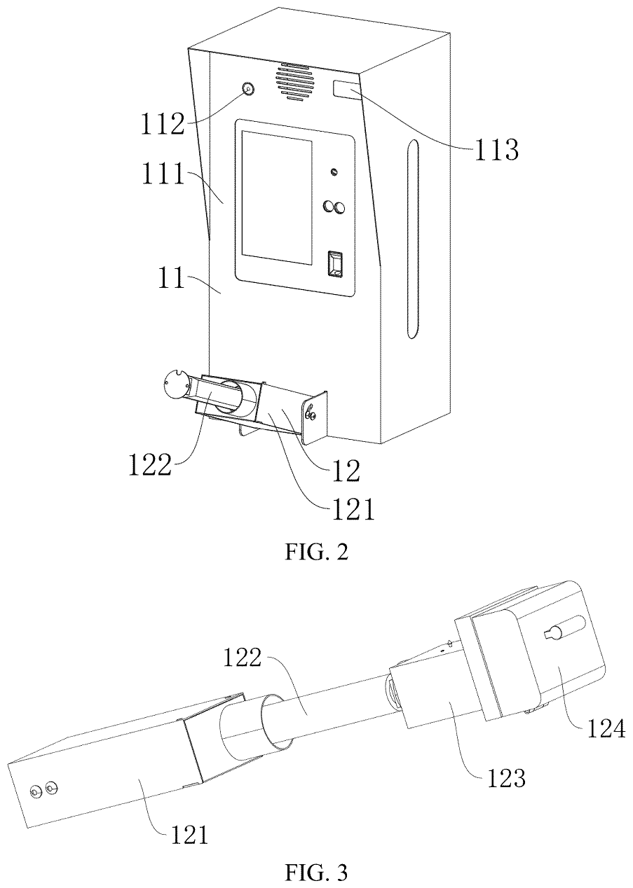

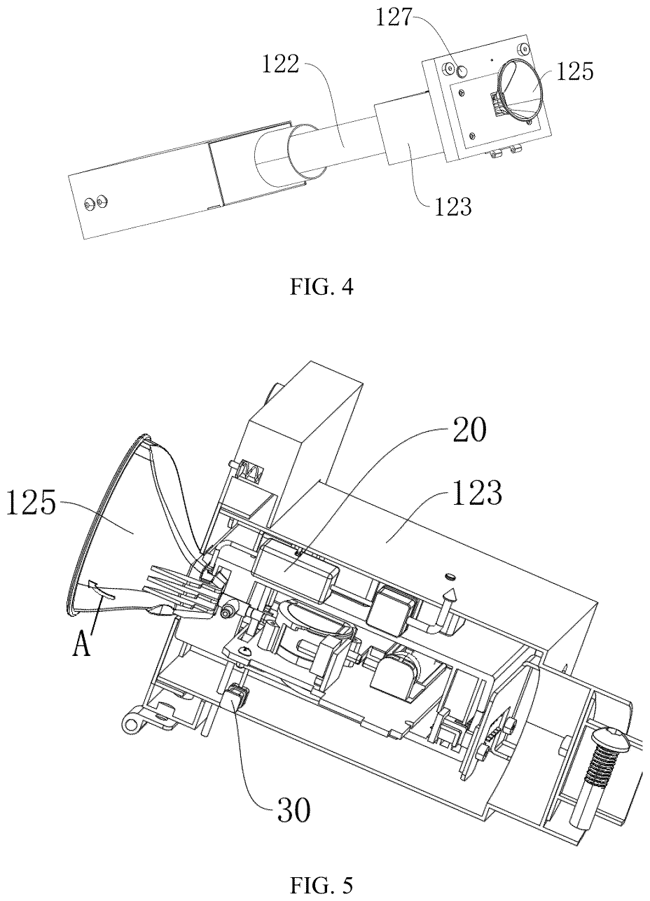

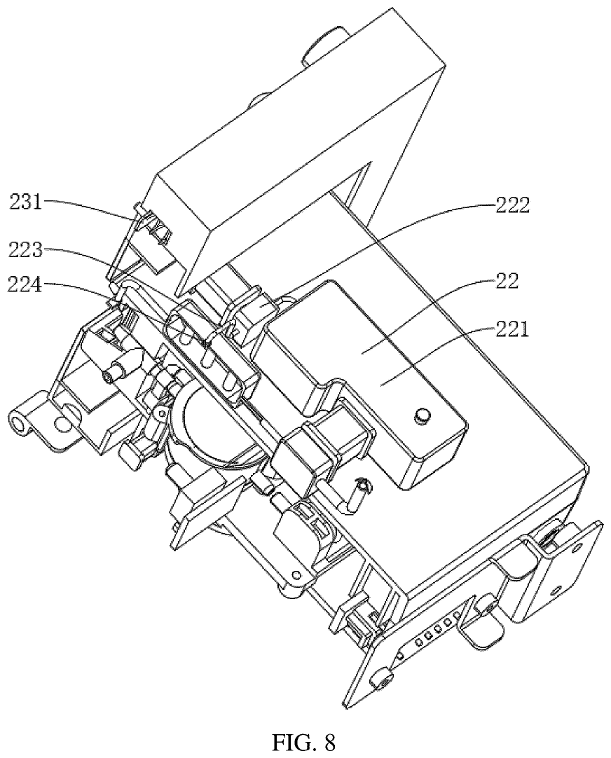

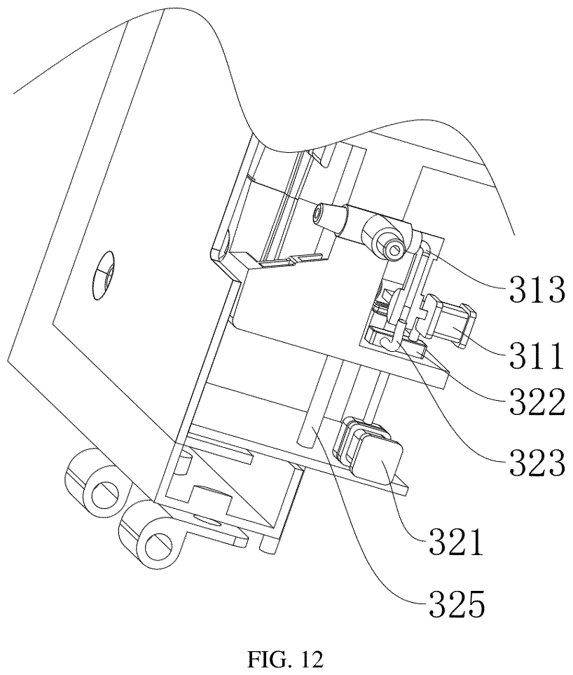

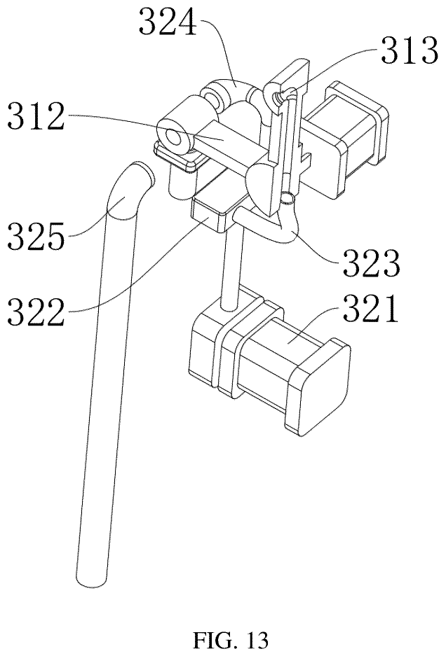

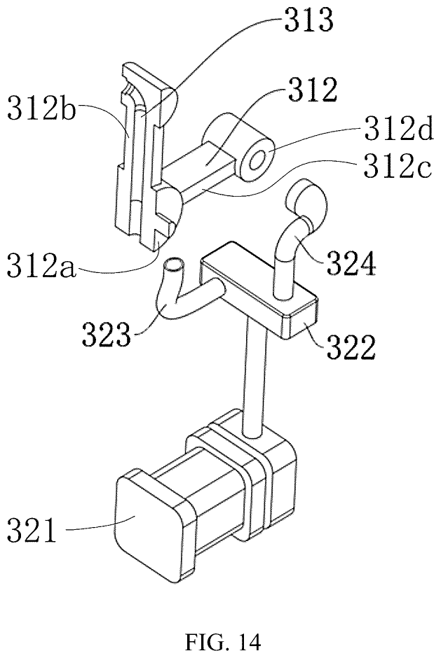

As shown in FIGS. 1 to 15, in some embodiments of the present disclosure, a retractable alcohol tester with intelligent recognition includes a main body 10, a cleaning assembly 20 and a re-cleaning assembly 30. The main body 10 includes a machine body assembly 11 provided with a detection assembly 12 adjacent to the bottom thereof. The cleaning assembly 20 includes an air heating component 21 arranged in the detection assembly 12, a steaming assembly 22 arranged above the air heating component 21, and a triggering assembly 23 arranged at the top of the inner cavity of the detection assembly 12. The re-cleaning assembly 30 includes a shifting component 31 arranged at the bottom of the detection assembly 12, and a dividing assembly 32 arranged below the shifting component 31.

In the embodiments, after the machine body assembly 11 recognizes a body size of a tested individual, the detection assembly 12 moves approach the tested individual to perform an alcohol detection. After achieving the alcohol detection, the air heating component 21 blows hot air into the detection assembly 12, the steaming assembly 22 injects water into the air heating component 21 to form steam, wherein the shifting component 31 is used to intercept the pipelines of the detection, and the dividing assembly 32 is used to clean the pipelines of the detection.

In an embodiment of the present disclosure, before the detection, a facial recognition of the tested individual is performed via a camera in the machine body assembly 11, so that the identity of the tested individual is accurately acquired by the tester. Subsequently, the detection part of the detection assembly 12 moves toward the individual's mouth to prepare for alcohol concentration detection. The detection component 123 includes an alcohol sensor. When the individual exhales, the alcohol sensor can detect the alcohol content in the exhaled gas and transmits the data to the machine body assembly 11 for analysis. During detection, the camera of the detection assembly 12 can record the testing process for subsequent verification and recording. After achieving the detection, the air heating component 21 of the cleaning assembly 20 starts operating and producing high-temperature hot air to disinfect the inner cavity of the detection assembly 12, so as to prevent the spread of bacteria and residual alcohol. At the same time, the steaming assembly 22 operates to inject water into the air heating component 21 to combine with the hot air for forming steam, further enhancing the disinfection effect. After achieving the detection, the shifting component 31 of the re-cleaning assembly 30 rotates downward to trigger the dividing assembly 32. A blowing part of the dividing assembly 32 starts operating, and a diversion part of the dividing assembly 32 divides the blowing air into two streams. One stream of blowing air is blown into the blowing part of the detection assembly 12 so as to flush the pipeline, while the other stream of blowing air enters the connection part of the shifting component 31 to ensure the cleanliness of the internal air, thereby preventing residual alcohol and the accumulation of bacteria. The design of the entire tester aims to provide a fast, accurate and hygienic alcohol detection process for ensuring the long-term stable operation of the tester and the health of tested individuals.

Referring again to FIGS. 1 to 6, the machine body assembly 11 includes a main frame 111.

A first camera 112 is fixed on the top of the front of the main frame 111, and an LED panel 113 is fixed to the top of the front of the main frame 111.

With reference to FIGS. 3 to 6, the detection assembly 12 includes an electric drive component 121 hinged at the bottom of the front side of the main frame 111, an electric push rod 122 mounted in the electric drive component 121, a detection component 123 connected to the top end of the electric push rod 122, a sealing cover 124 hinged on the front side of the detection component, and a blowing nozzle 125 detachably connected to the front side of the detection component 123. The detection component 123 is provided with a locking component 126 arranged above the blowing nozzle 125. A second camera 127 is arranged at the top of the front side of the detection component 123.

It should be noted that the first camera 112 and the second camera 127 are both equipped with facial recognition and AI (artificial intelligence) modules (not shown in the drawings, which are common technical software and hardware parts in the prior art without detail description herein). The cameras are provided to recognize and record the tested individual, so that the electric drive component 121 and electric push rod 122 are guided to operate and approach the breathing mouth of the tested individual.

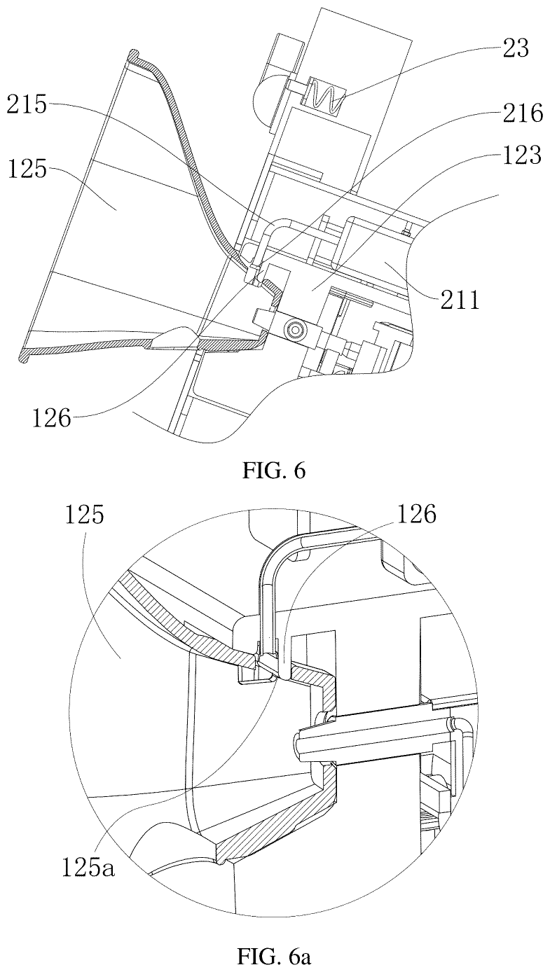

With reference to FIG. 6, in a configuration, the blowing nozzle 125 is provided with an arc-shaped groove 125a in contact with the locking component 126 adjacent to the bottom of the blowing nozzle 125. In the configuration, the blowing nozzle 125 can be limited by the mating between the arc-shaped groove 125a and the locking component 126, when the blowing nozzle 125 is aligned to the detection component 123. Furthermore, the blowing nozzle 125 can be detached by lifting and pulling along an outward direction, with reference to an identification arrow A on the blowing nozzle 125 as shown in FIG. 5.

In the embodiment, before an alcohol detection, the first camera 112 firstly performs facial recognition to confirm and record the identity of the tested individual. After successful recognition, the electric drive component 121 can be controlled by the AI module and drive the electric push rod 122 to push the detection component 123 forward until the blowing nozzle 125 contacts the tested individual's breathing mouth. Subsequently, the sealing cover 124 is opened and the tested individual blows into the blowing nozzle 125 to start the detection. At this time, the second camera 127 starts to record facial images during the detection process to ensure the legality and accuracy of the test. An alcohol sensor in the detection component 123 starts to analyze the alcohol content in the exhaled gas. If the detection result is normal (i.e., none alcohol is detected), the LED panel 113 emits green light; if a little of existing alcohol is detected, the LED panel 113 emits orange light; and if high concentration of the alcohol is detected, the LED panel 113 emits red light and alerts the detection result.

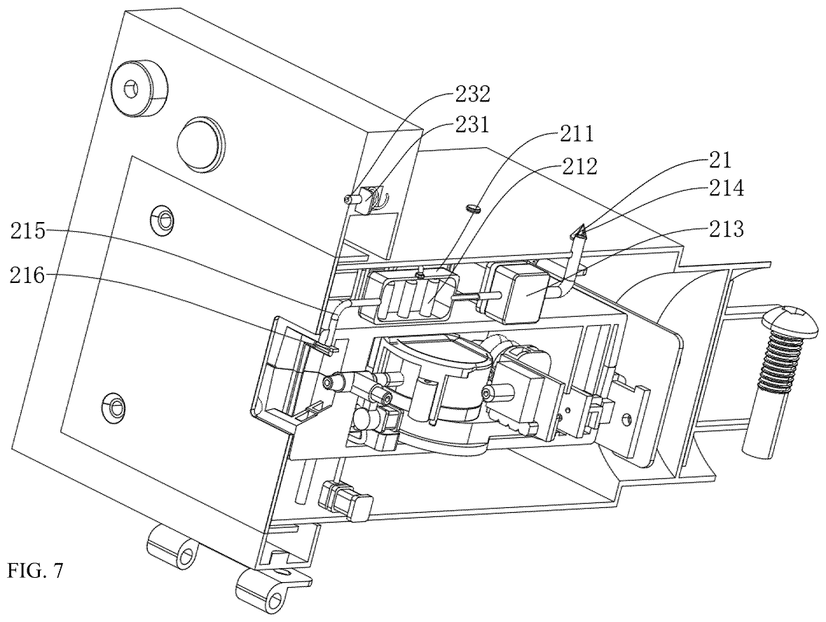

Moreover, referring again to FIGS. 5 to 9, the air heating component 21 includes a thermal insulation gas buffer 211 fixed to the top of the inner cavity of the detection component 123. An electric heating element 212 is mounted in the gas buffer 211, and a first fan 213 (such as an induced draft fan) is connected to the rear side of the gas buffer 211. An air inlet hood 214 is connected to the inlet of the first fan 213, where the air inlet hood 214 is fixed on the top surface of the detection component 123. Referring to FIG. 7, the pipe connecting the air inlet hood and the first fan 213 pass though the top surface of the detection component 123. In addition, one end of an air pipe 215 is connected to the front side of the gas buffer 211, and a blowing hood 216 is connected to the other end of the air pipe 215, where the blowing hood 216 is arranged above the blowing nozzle 125.

The steaming assembly 22 includes a water tank 221, a water pump 222, a water pipe 223 and a check valve 224. The water tank 221 is fixed to the top of the inner cavity of the detection component 123. The water pump 222 is connected to the outlet of the water tank 221, and fixed to the top of the inner cavity of the detection component 123. The water pipe 223 is provided with an upper end connected to the outlet of the water pump 222, and a lower end connected to the top of the gas buffer 211. The check valve 224 is mounted at a position where the gas buffer 211 is connected to the water pipe 223.

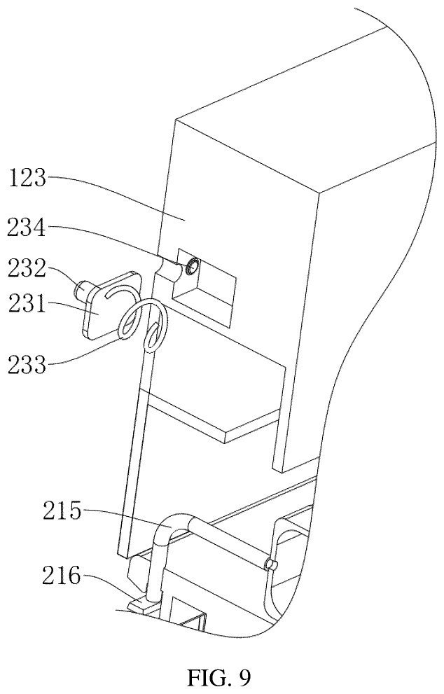

Referring to FIG. 9, the triggering assembly 23 includes a sliding plate 231 slidably arranged at the top of the inner cavity of the detection component 123. A pressing rod 232 is connected to the outward side (or the outward surface) of the sliding plate 231 facing the outer side of the detection component 123, where the outer end of the pressing rod 232 is in contact with the bottom of the sealing cover 124. One end of a spring 233 is connected to the inward side (or the inward surface) of the sliding plate 231 facing the inner side of the detection component 123, and the other end of the spring 233 is fixedly connected to the middle part of the inner cavity of the detection component 123. A first button 234 is mounted to the top of the inner cavity in the detection component 123, and the first button 234 can be in contact with the outer side of the sliding plate 231 as the sliding plate 231 is push by the spring 233.

In an embodiment, the first button 234 can be pressed by the sealing cover 124 in its closed position. It should be noted that the first button 234 is a component that controls the operation of the electric heating element 212, so as to control preheating as the sealing cover 124 is opened for detection, thereby improving the efficiency of subsequent cleaning and disinfection.

In an embodiment of the present disclosure, the top of the water tank 221 is provided with a water inlet arranged at the top of the detection component 123.

In the embodiment of the present disclosure, before the detection and after the sealing cover 124 is opened, the pressure on the pressing rod 232 is released, and then the spring 233 rebounds to push the sliding plate 231 to move and trigger the first button 234, causing the electric heating element 212 to start preheating. After achieving the detection, when the second camera 127 detects that the tested individual has moved away, the first fan 213 starts to suck air into the gas buffer 211, and the air is heated by the electric heating element 212 to form high-temperature hot air. The hot air passes through the air pipe 215 and blowing hood 216 to perform high-temperature disinfection on the blowing nozzle 125. At the same time, the water pump 222 starts to draw water from the water tank 221 and delivering it to the gas buffer 211 through the water pipe 223, where it combines with the hot air to form steam, further enhancing the disinfection effect. The water enters the gas buffer 211 through the check valve 224, ensuring that the steam flows in only one direction and preventing backflow.

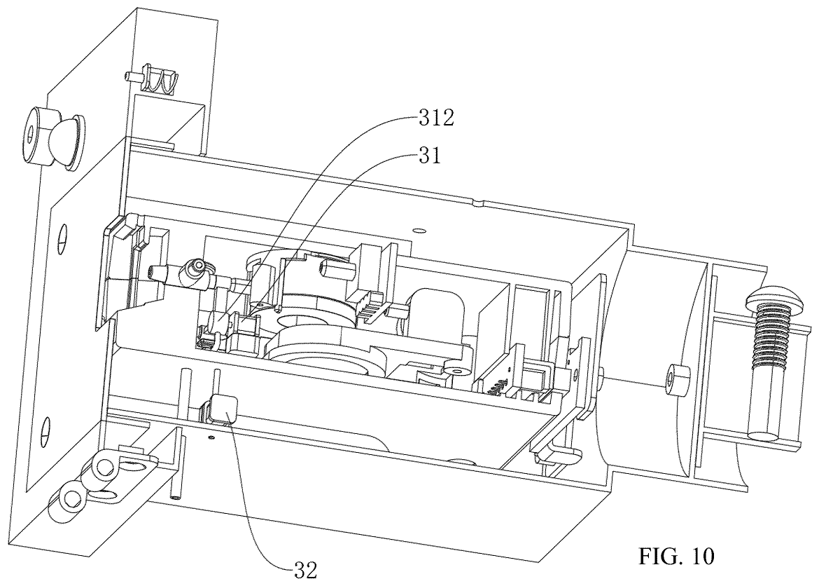

In an embodiment of the present disclosure, referring to FIGS. 10 to 15, the shifting component 31 includes a motor 311 mounted at the bottom of the inner cavity of the detection component 123 and a rotating frame 312 having a base portion 312a connected to the output shaft of the motor 311, wherein the base portion 312a is provided with a connection port. In addition, the rotating frame 312 includes two arms 312a, 312b respectively fixed to the base portion 312a. The first arm 312b includes a blind hole at an end away from the base portion 312a, and an L-shaped pipe channel jointing the blind hole to the connection port of the base portion 312a. An L-shaped pipe (not shown) is provided in the L-shaped pipe channel. The second arm 312c includes a sleeve portion 312d (or an outer ring) having a though hole at an end away from the base portion 312a.

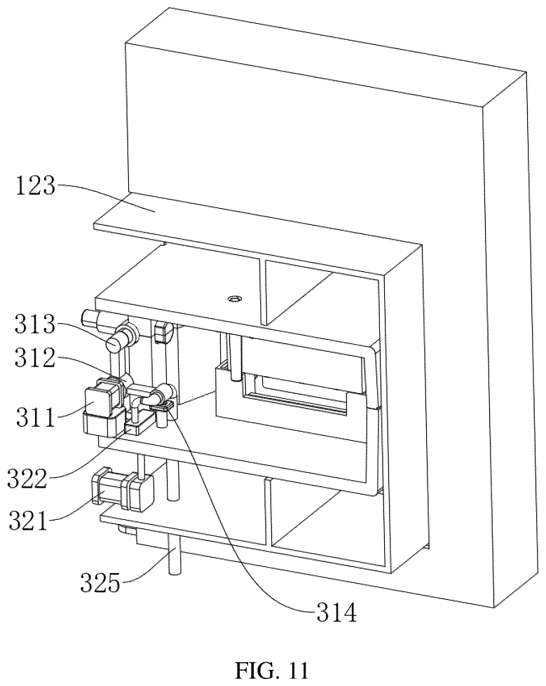

In an embodiment, a second button 314 is provided to mount at the bottom of the inner cavity of the detection component 123, where the sleeve portion or the outer ring of the rotating frame 312 can be in contact with the second button 314, as shown in FIG. 11.

In an embodiment, the dividing assembly 32 includes a second fan 321 mounted at the bottom of the inner cavity of the detection component 123, and a three-way component 322 having three ports, wherein the first port (such as an inlet port) is connected to the outlet of the second fan 321. Specifically, the three-way component 322 is provided with a gas chamber joining the three ports. A soft pipe 323 is provided with one end connected to the second port (such as an outlet port) of the three-way component 322, and the other end connected to the connection port of the base portion 312a. A blowing pipe 324 is provided with an inlet end connected to the third port (such as another outlet port) of the three-way component 322, and an outlet end for accessing to one side of the sleeve portion 312d of the rotating frame 312. An exhaust pipe 325 is provided with an inlet end for matching the other side of the sleeve portion 312d, and an outlet extending out of the detection component 123 from the bottom.

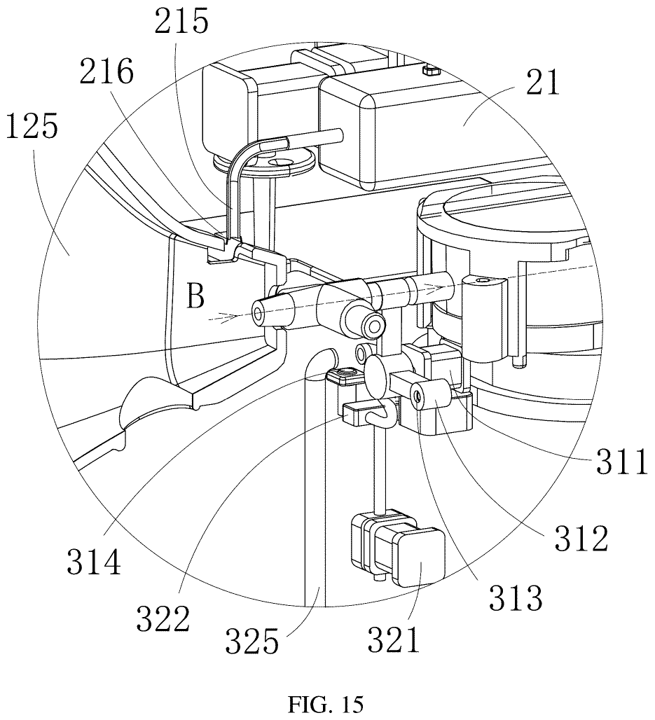

In the embodiment, the rotating frame is driven by the motor to rotate between a first position for alcohol detection process and a second position for cleaning the nozzle. A hollow pipe (not shown) is provided in the hole of the sleeve portion 312d for connecting two pipe components when the rotating frame 312 rotates to the first position. In the embodiment, one pipe component is connected to the blowing nozzle 125, and the other pipe component is connected to the detection component 123. Furthermore, the inner diameter of each of the hollow pipes matches the inner diameter of the pipeline in the detection component 123, so as to allow the gas from the blowing nozzle 125 to be delivered into the interior of the detection component 123 through the hollow pipes, with reference to FIG. 15.

In the embodiment, the blowing pipe 324 and the exhaust pipe 325 are arranged on the both sides of the rotating frame 312, so that the hollow pipe connects the two pipes 324, 325 and the L-shaped pipe connects the pipe component connected to the blowing nozzle 125 and the soft pipe 323, when the rotating frame 312 rotates to the second position.

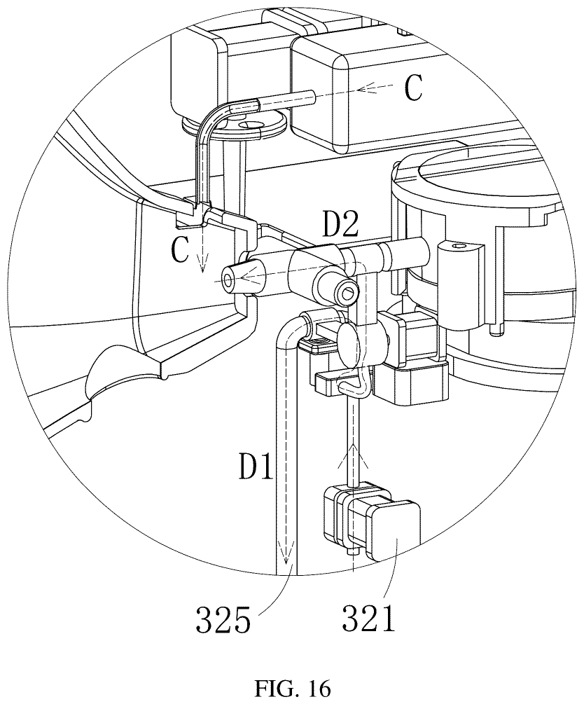

It should be noted that, the blowing pipe 324 and the exhaust pipe 325 are both hard pipes, and the hollow pipe in the rotating frame 312 can be soft, so as to reduce or prevent an offset between the blowing pipe 324 and the exhaust pipe 325 due to the contact of the pipes when the hollow pipe is rotated to a position between the blowing pipe 324 and the exhaust pipe 325 (where the rotating frame 312 rotates to the second position, with reference to FIG. 16). Both the blowing pipe 324 and the exhaust pipe 325 can be made of brass, so as to improve the rust-proof and antibacterial properties.

In an embodiment of the present disclosure, the second button 314 is a switch for controlling the second fan 321 to open or close.

In the embodiment of the present disclosure, during the detection, the gas received by the blowing nozzle 125 is guided into the interior of the detection component 123 through the hollow pipe of the rotating frame 312 for detection. After achieving the detection, the motor 311 drives the rotating frame 312 to rotate to a second position, so that the hollow pipe in the second arm 312c of the rotating frame 312 is aligned and connected to the blowing pipe 324, and the first arm 312b triggers the second button 314. At this time, the L-shaped pipe channel 313 is aligned and connected to the pipeline of the blowing nozzle 125. The second button 314 controls the second fan 321 to blow outer air into the three-way component 322. The three-way component 322 divides the air inducted by the second fan 321 into two streams. One stream enters the blowing pipe 324 and blows into the hollow pipe in the rotating frame 312 for flushing, until discharging through the exhaust pipe 325, with reference to the first gas cleaning path D1 in FIG. 16. The other stream enters the L-shaped pipe channel 313 through the soft pipe 323 and blows into the pipeline connected to the blowing nozzle 125 to flush the interior of the pipeline, thereby achieving the cleaning and disinfection of the tester, with reference to the first gas cleaning path D2 in FIG. 16. In this design, the tester can be deeply cleaned and disinfected after each detection, thereby effectively preventing the risk of cross-infection and avoiding residual alcohol gas in the pipelines.

In the embodiment of the present disclosure, the complete working procedure of the alcohol tester is as follows. Before the detection, the first camera 112 performs facial recognition to confirm and record the identity of the tested individual. Subsequently, the electric push rod 122 pushes the detection component 123 forward until the blowing nozzle 125 approach the tested individual's mouth. Afterward, the sealing cover 124 is opened, and the tested individual blows into the blowing nozzle 125 to start the detection. With reference to FIG. 15, the rotating frame is already rotated to the first position for gas detection, so that the blowing gas from the nozzle 125 can be transmitted into the detection component 123 along the gas detection path B. Subsequently, the second camera 127 starts to record facial images in the detection process. If the detection result is normal (i.e., none alcohol is detected), the LED panel 113 emits green light; if a little of existing alcohol is detected, the LED panel 113 emits orange light; and if high concentration of the alcohol is detected, the LED panel 113 emits red light and alerts the detection result.

In addition, after opening the sealing cover 124, the pressure on the pressing rod 232 is released, and then the spring 233 rebounds to push the sliding plate 231 to move and trigger the first button 234, causing the electric heating element 212 to start preheating. After achieving the detection, if the second camera 127 detects that the tested individual has moved away, the first fan 213 starts to suck air into the gas buffer 211, and the air is heated by the electric heating element 212 to form high-temperature hot air. The hot air passes through the air pipe 215 and blowing hood 216 to perform high-temperature disinfection on the blowing nozzle 125. At the same time, the water pump 222 starts to draw water from the water tank 221 to the gas buffer 211 through the water pipe 223, where the water is evaporated to vapor to combine with the hot air to form a hot steam to enter the blowing nozzle 125 along a hot steam disinfecting path C, in order to enhance the disinfection effect. Simultaneously, the motor 311 drives the rotating frame 312 to rotate to the second position for gas cleaning, so that the hollow pipe in one arm of the rotating frame 312 is aligned and connected to the blowing pipe 324, and the other arm of the rotating frame 312 trigger the second button 314. At this time, the L-shaped pipe channel 313 is aligned and connected to the pipeline component of the blowing nozzle 125. The second button 314 controls the second fan 321 to blow the outer gas into the three-way component 322. The three-way component 322 divides the air inducted by the second fan 321 into two streams. One stream enters the blowing pipe 324 and blows into the hollow pipe in the rotating frame 312 for flushing, until discharging through the exhaust pipe 325, with reference to the first gas cleaning path D1 in FIG. 16. The other stream enters the L-shaped pipe channel 313 through the soft pipe 323 and blows into the pipeline connected to the blowing nozzle 125 to flush the interior of the pipeline, thereby achieving the cleaning and disinfection of the tester, with reference to the first gas cleaning path D2 in FIG. 16.

The above description is only a preferred embodiment of the disclosure. The disclosure is not limited to the above embodiments. As long as the same means can achieve the technical effects of the disclosure, any modifications, equivalent substitutions, improvements, etc., made within the spirit and principles disclosed herein should be included within the scope of the disclosure. Various modifications and variations can be made to the technical solution and/or implementation method within the scope of the disclosure.

LIST OF REFERENCE SIGNS

| | | | |
|---|---|---|---|
| 10 | main body | 23 | triggering assembly |
| 11 | machine body assembly | 231 | sliding plate |
| 111 | main frame | 232 | pressing rod |
| 112 | first camera | 233 | spring |
| 113 | LED panel | 234 | first button |
| 12 | detection assembly | 30 | re-cleaning assembly |
| 121 | electric drive component | 31 | shifting component |
| 122 | electric driving rod | 311 | motor |
| 123 | detection component | 312 | rotating frame |
| 124 | sealing cover | 312a | base portion |
| 125 | blowing nozzle | 312b | first arm |
| 125a | arc-shaped groove | 312c | second arm |
| 126 | locking component | 312d | sleeve portion |
| 127 | second camera | 313 | L-shaped pipe channel |
| 20 | cleaning assembly | 314 | second button |
| 21 | air heating component | 32 | dividing assembly |
| 211 | thermal insulation gas buffer | 321 | second fan |
| 212 | electric heating element | 322 | three-way component |
| 213 | first fan | 323 | soft pipe |
| 214 | air inlet hood | 324 | blowing pipe |
| 215 | air pipe | 325 | exhaust pipe |
| 216 | blowing hood | A | identification arrow |
| 22 | steaming assembly | B | gas detection path |
| 221 | water tank | C | hot steam disinfecting path |
| 222 | water pump | D1 | first gas cleaning path |
| 223 | water pipe | D2 | second gas cleaning path |
| 224 | check valve | | |

1. An alcohol tester, comprising:
a main body having a machine body assembly provided with a detection assembly for an alcohol detection at the bottom;
a cleaning assembly comprising an air heating component arranged in the detection assembly, a steaming assembly arranged above the air heating component, and a triggering assembly arranged at the top of an inner cavity of the detection assembly; and
a re-cleaning assembly comprising a shifting component arranged at the bottom of the detection assembly, and a dividing assembly arranged below the shifting component;
wherein, before an alcohol detection, the machine body assembly recognizes a body size of a tested individual and the detection assembly moves approach the tested individual; and
wherein, after the alcohol detection, the air heating component blows hot air into the detection assembly, and the steaming assembly injects water into the air heating component to form steam, the shifting component intercepts pipelines of the alcohol detection, and the dividing assembly clean the pipelines of the alcohol detection.

2. The alcohol tester according to claim 1, wherein the machine body assembly comprises a main frame,
a first camera fixed to the front top of the main frame, and
an LED panel fixed to the front top of the main frame.

3. The alcohol according to claim 2, wherein the detection assembly comprises:
an electric drive component hinged at the bottom of front side of the main frame;
an electric push rod mounted in the electric drive component; and
a detection component connected to the top end of the electric push rod;
wherein, the front side of the detection component is hinged with a sealing cover and a blowing nozzle is detachably connected to the front side of the detection component, and
wherein, the detection component is provided with a locking component arranged above the blowing nozzle and a second camera is mounted at the top of the front side of the detection component.

4. The alcohol tester according to claim 3, wherein the air heating component comprises:
a thermal insulation gas buffer fixed to the top of an inner cavity of the detection component;
at least one electric heating element mounted in the thermal insulation gas buffer;
a first fan having an inlet and an outlet connected to the rear side of the thermal insulation gas buffer;
an air inlet hood connected to the inlet of the first fan; and
a blowing hood arranged above the blowing nozzle;
wherein, the air inlet hood is fixed on the top surface of the detection component, the front side of the thermal insulation gas buffer is connected to one end of an air pipe, and the blowing hood is connected to the other end of the air pipe.

5. The alcohol tester according to claim 4, wherein the steaming assembly comprises
a water tank fixed to the top of the inner cavity of the detection component,
a water pump connected to an outlet of the water tank,
a water pipe connecting the water pump with the thermal insulation gas buffer, and
a check valve mounted at a position where the thermal insulation gas buffer is connected to the water pipe.

6. The alcohol tester according to claim 3, wherein the triggering assembly comprises:
a sliding plate having an outward surface facing an outer side of the detection component and an inward surface facing an inner side of the detection component;
a pressing rod connected to the outward surface of the sliding plate;
a spring having one end connected to the inward surface of the sliding plate; and
a first button mounted at the top of the inner cavity of the detection component;
wherein, the sliding plate is arranged at the top of the inner cavity of the detection component, and an outer end of the pressing rod extends out of the detection component in contact with the bottom of the sealing cover, the other end of the spring is fixed to the inner cavity of the detection component, and the first button is accessible to the outward surface of the sliding plate.

7. The alcohol tester according to claim 1, wherein the shifting component comprises:
a rotating frame comprising a base portion, a first arm, and a second arm having an sleeve portion for connecting pipelines for alcohol detection;
a motor having an output shaft connected to the base portion of the rotating frame;
an L-shaped pipe channel connecting the outer of the first arm to the base portion; and
a second button mounted at the bottom of the inner cavity of a detection component in the detection assembly,
wherein, the second button is triggered by the second arm to clean the sleeve portion, and wherein the motor is mounted at the bottom of the inner cavity of the detection component.

8. The alcohol tester according to claim 7, wherein the dividing assembly comprises:
a three-way component having an inlet port, a first outlet port and a second outlet port;
a second fan connected to the inlet port of the three-way component;
a soft pipe having one end connected to the first outlet port of the three-way component, and the other end connected to the L-shaped pipe channel of the rotating frame;
a blowing pipe having an inlet end connected to the second outlet port of the three-way component, and an outlet end for accessing to one side of the sleeve portion of the rotating frame; and
an exhaust pipe having an inlet end for accessing to the other side of the sleeve portion of the rotating frame, and an outlet extending out of the detection component from the bottom;
wherein the second fan is mounted at the bottom of the inner cavity of the detection component.

9. The alcohol tester according to claim 8, wherein the blowing pipe and the exhaust pipe are hard pipes made of brass.

10. The alcohol tester according to claim 8, wherein the second button is a switch for controlling an operation of the second fan.

11. The alcohol tester according to claim 7, wherein the rotating frame is provided with a hollow pipe in the sleeve portion, and the inner diameter of the hollow pipe matches with inner diameters of pipelines in the detection component.

12. An alcohol tester, comprising:
a main body having a machine body assembly provided with a detection assembly for an alcohol detection at the bottom; and
a re-cleaning assembly comprising a shifting component arranged at the bottom of the detection assembly, and a dividing assembly arranged below the shifting component;
wherein, after the alcohol detection, the shifting component intercepts pipelines of the alcohol detection, and the dividing assembly clean the pipelines of the alcohol detection; and
wherein, the shifting component comprises:
a rotating frame comprising a base portion, a first arm, and a second arm having an sleeve portion for connecting pipelines of the alcohol detection;
a motor having an output shaft connected to the base portion of the rotating frame;
an L-shaped pipe channel connecting the outer of the first arm to the base portion; and
a button mounted at the bottom of the inner cavity of a detection component in the detection assembly, wherein the button is triggered by the second arm of the rotating frame rotated by the motor to clean the sleeve portion.

13. The alcohol tester according to claim 12, wherein,
the rotating frame is provided with a hollow pipe in the sleeve portion, and the inner diameter of the hollow pipe matches with inner diameters of pipelines in the detection component, and wherein,
the hollow pipe connects the pipelines when the rotating frame is driven by the motor to rotate to a position for alcohol detection.

14. The alcohol tester according to claim 12, wherein the dividing assembly comprises:
a three-way component having an inlet port, a first outlet port and a second outlet port;
a fan connected to the inlet port of the three-way component;
a soft pipe having one end connected to the first outlet port of the three-way component, and the other end connected to the L-shaped pipe channel of the rotating frame;
a blowing pipe having an inlet end connected to the second outlet port of the three-way component, and an outlet end for accessing to one side of the sleeve portion of the rotating frame; and
an exhaust pipe having an inlet end for accessing to the other side of the sleeve portion of the rotating frame, and an outlet extending out of the detection component from the bottom;
wherein the fan is mounted at the bottom of the inner cavity of the detection component.

15. The alcohol tester according to claim 14, wherein the blowing pipe and the exhaust pipe are hard pipes made of brass.
